(19) United States
(12) Patent Application Publication
(10) Pub. No.: US 2026/0335535 A1
(43) Pub. Date: Oct. 29, 2026

(54) MOBILE IV STAND

(71) Applicant: Thomas Jefferson University, Philadelphia, PA (US)

(72) Inventors: Joseph Braverman, Philadelphia, PA (US); Maitri Doshi, Philadelphia, PA (US); Rae Monane, Philadelphia, PA (US); David Gordon, Philadelphia, PA (US)

(21) Appl. No.: 19/114,248

(22) PCT Filed: Sep. 25, 2023
(86) PCT No.: PCT/US2023/075015

§ 371 Date: Mar. 21, 2025

Related U.S. Application Data
(60) Provisional application No. 63/376,842, filed on Sep. 23, 2022.

(51) Int. Cl. A61M 5/14

(52) U.S. Cl. CPC A61M 5/1415; A61M 2209/084

(57) ABSTRACT

Aspects of the present invention relate to a mobile IV stand including a base having a top surface and a bottom surface, the base further including a central region, a front end, and a back end defined by two legs extending from the central region, at least one wheel on the bottom surface of the base underneath each leg, and at least one wheel on the bottom surface of the base underneath the central region, an IV pole extending from the top surface of the base, and a handle pole extending from the top surface of the base, wherein the handle pole is separate and independent from the IV pole, and wherein the handle pole moves along at least one axis between at least a first and second lockable position.

### CROSS-REFERENCE TO RELATED APPLICATIONS

This application claims the benefit of priority to U.S. Patent Application No. 63/376,842, filed on Sep. 23, 2022, the contents of which are incorporated by reference herein as if set forth in its entirety.

### BACKGROUND OF THE INVENTION

Increasingly, evidence has suggested patients regularly ambulate following surgery, as evidence has shown that ambulation improves patient outcomes postoperatively (Rothman et al., “The impact of early ambulation in the pediatric postoperative appendectomy patient.” Journal of pediatric surgical nursing 5.3 (2016): 70-75). Today, much of this patient activity requires the time and attention of a nurse on the service. A team member or support person may need to tow additional medical equipment along with the patient once they get out of bed to walk. Ambulating can be a cumbersome, difficult experience. Patients need to remain tethered to an IV pole, carrying required medication, pumps, and machinery.

While IV poles can be found in every patient's room, their form has barely changed from their conception in 1904. The model of care at that time did not encourage patients to move about the hospital, as is increasingly commonplace today. Moreover, the scope and types of equipment fastened to this pole have changed considerably over the last century, and it no longer sits motionless at an immobile patient's bedside. Nurses and patients report issues with almost every element of the current IV pole form, which can affect care delivery. No existing IV system on the market has been designed to accommodate mobility, while also containing the core pumps and features, to enable patients to ambulate without additional equipment.

There are no major medical supply distributors that carry a market-available item that meets these needs today. A mobile IV stand is needed to serve both pediatric and adult populations, with an intuitive and safe design that is exceptionally difficult to tip over while ambulating through doorways, over thresholds, around corners and in tight spaces (like a hospital patient-room bathroom).

Thus, there is the need in the art for a mobile IV stand designed to accommodate mobility of the subject. The present invention meets this need.

### SUMMARY OF THE INVENTION

Aspects of the present invention relate to a mobile IV stand including a base having a top surface and a bottom surface, the base further including a central region, a front end, and a back end defined by two legs extending from the central region, at least one wheel on the bottom surface of the base underneath each leg, and at least one wheel on the bottom surface of the base underneath the central region, an IV pole extending from the top surface of the base, and a handle pole extending from the top surface of the base, wherein the handle pole is separate and independent from the IV pole, and wherein the handle pole moves along at least one axis between at least a first and second lockable position.

In some embodiments, the handle is connected to the base at a hinging joint. In some embodiments, the hinging joint may be releasably secured by one or more locking mechanisms to arrest movement of the handle pole. In some embodiments, the one or more locking mechanisms includes at least one of a friction clamp, spring pin, ratchet, brake and piston.

In some embodiments, the mobile IV stand further includes one or more actuators positioned on the handle of the handle pole configured to releasably actuate the locking mechanism. In some embodiments, the one or more actuators include at least one of a button, switch and a trigger. In some embodiments, the handle pole is configured to pivot along the at least one axis between 0 degrees and 90 degrees relative to the IV pole.

In some embodiments, the mobile IV stand further includes one or more hand-grips attached to the handle pole. In some embodiments, the one or more hand-grips include a pair of vertical hand-grips and a pair of horizontal hand-grips. In some embodiments, the one or more hand-grips have knurling, debossed features, embossed features, slits, or material coverings such as foam or rubber.

In some embodiments, the at least one of the IV pole and handle pole include one or more telescoping mechanisms. In some embodiments, the one or more telescoping mechanisms are locked by one or more locking mechanisms. In some embodiments, the locking mechanisms include at least one of a clamp, cam lock, spring pin, latch, and piston. In some embodiments, each leg has a length between 1 inch and 24 inches. In some embodiments, the mobile IV stand further includes a brake configured to releasably lock at least one wheel.

In some embodiments, the IV pole includes one or more attachment elements for healthcare equipment. In some embodiments, the healthcare equipment is selected from the group consisting of: IV pumps, IV medication bags, foley catheter bags, oxygen tanks, power banks, and portable wound dressing suction units.

In some embodiments, at least one of the IV pole and the handle pole has a recess configured to at least partially house the opposing pole. In some embodiments, the two legs at least partially define a walking space.

Aspects of the present invention relate to a mobile IV stand including a base having a pair of opposing legs and a walking space therebetween, a plurality of wheels extending from a bottom surface of the base, an IV pole extending from a top surface of the base along a first axis, and a handle pole extending from the top surface of the base along a second axis, the handle pole connected to the base at a pivot point and configured to at least partially pivot into an area above the walking space.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 is an image of an exemplary embodiment of a mobile IV stand.

FIG. 2 is a schematic depicting a top-down view of a mobile IV stand.

FIG. 3 is a schematic of an exemplary hinging mechanism for the handle pole component of a mobile IV stand.

FIG. 4 is a schematic of the base portion and connections of the IV pole and handle pole to the base portion of a mobile IV stand.

FIG. 5 is a schematic of a handle portion connected to the handle pole of a mobile IV stand.

FIG. 6 depicts a mobile IV stand in a hospital environment with a human subject for scale.

FIG. 7 depicts a mobile IV stand in various healthcare scenarios accommodating for different use states including: ambulating in hallways, stowing within the cluttered bedside environment, and navigating tight spaces.

FIG. 8 shows a subject using a mobile IV stand with normal ambulation with foot ingress into a recessed portion of the base.

FIG. 9 shows a subject pulling on a mobile IV stand to display the resistance of the design to toppling over.

FIG. 10 depicts an existing IV Pole (left) and a mobile IV stand (right) according to aspects of the present invention.

FIG. 11 depicts side views of a mobile IV stand with force diagram overlays and the resultant loads.

FIG. 12 displays the results for a competitive analysis between an existing IV Pole, a prior study IV pole, and a mobile IV stand.

FIG. 13 depicts a test method for a mobile IV stand including gait analysis of the subject with a control of an existing IV pole.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of IV stands. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Mobile IV Stand System

Generally, aspects of the invention include a mobile IV stand with a handle that is attached to a handle pole separate and independent from a pole that IVs are hung. The handle pole attaches to a pivot point with a series of locking points at the base so that the handle can be moved away from the IV pole into a position where a patient can more easily walk with the IV pole. When collapsed, the handle on the handle pole is still accessible and the overall footprint of the unit is smaller to allow for stowing or stationary use in a crowded hospital room. The handle pole may be locked into place at various angles using a spring-loaded internal piston-driven mechanism or other locking mechanisms. The locking mechanism may be actuated at or close to the handle. The locking mechanism may include several locking channels at the base of the stand to allow for the pivoting handle pole to be locked into multiple pre-set positions and also limit the pivot travel so it remains within a certain boundary that does not increase the risk for tipping. The pivoting handle pole can exist in multiple configurations including a version where it nests into the non-pivoting pole via a recess in the non-pivoting pole. Another iteration includes where the pivoting handle pole and non-pivoting IV pole do not recess into one another and are parallel to one another when in the non-deployed state. Further, the shape of the base component may be specifically designed so that a patient walking with the mobile IV stand has a open or recessed space for their feet to go, thereby limiting the tripping hazard when ambulating with the mobile IV stand. The specific location of the wheels and the center of gravity of the base allows the handle mechanism to function safely and not lead to a tipping risk.

The disclosed invention addresses the immense need for a better ambulation solution for those who require an IV in the hospital. Disclosed is a mobile IV stand that addresses the mobility and stability issues of existing IV poles. Additionally, the disclosed invention promotes ease of patient navigation, accommodating adult and pediatric patient populations, while also addressing the array of hazards associated with ambulation with the standard IV pole.

In many cases, IV poles are not designed for patient ambulation as the poles may tip over easily, and the subject's feet may interfere with the base of the IV pole. The disclosed invention aims to address these shortcomings with the addition of a separate pole with handle for control and movement of the base, and a base design allowing ingress of the feet into a recessed portion of the base during normal walking gait.

In some embodiments, the mobile IV stand is designed to accommodate existing healthcare equipment. This includes, but is not limited to, IV pumps, IV medication bags, foley catheter bags, oxygen tanks, power banks, portable wound dressing suction units.

In some embodiments, the mobile IV stand is designed with postoperative patient limitations in mind while improving surgical outcomes. In some embodiments, the mobile IV stand is designed with the flexibility to adjust or scale dimensionally for patients.

In some embodiments, the mobile IV stand is designed to be modular, to enable fast and easy replacement or repair of components. In some embodiments, attachments to the mobile IV stand may provide users with space, hangers or attachment points for personal belongings.

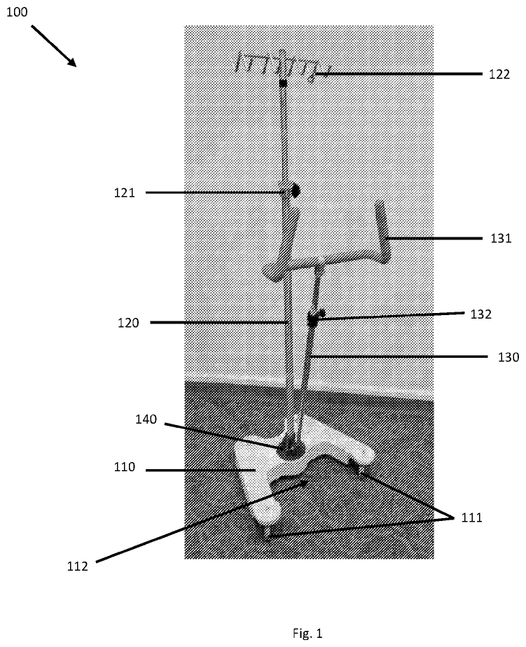

Referring now to FIG. 1, shown is a mobile IV stand 100 generally comprising a base 110, an IV pole 120, and a handle pole 130. In some embodiments, one or more pole connectors 140 may be used to attach IV pole 120 and/or handle pole 130 to base 110.

For example, pole connector 140 may separately engage both IV pole 120 and handle pole 130 for ergonomic movement of mobile IV stand 100.

Base 110 may include one or more wheels 111 on the bottom surface of base 110. Wheels 111 may be any type of wheels permitting movement in any desired direction along a floor surface. Wheels 111 may be lockable to prohibit movement. Exemplary wheels 111 may be caster wheels. In some embodiments, the locking of wheels 111 may be actuable via a foot-brake mounted on base 110 and/or a hand-brake mounted on handle 131 and/or handle pole 130.

Base 110 of mobile IV stand 110 differs from typical IV poles in that the center of the system's base is positioned slightly towards the user, to provide nooks or recessed portions for the subjects' feet. This provides a recessed portion or inset cavity in the base such that the subject's feet will not interfere or collide with the base during normal walking gait.

In some embodiments, the base comprises two lateral wheel stabilizers, a central pole, and two front stabilizers. In some embodiments, the base comprises at least one roller wheel forming a wheelbase such that the base may roll on the floor. In some embodiments, the base and arrangement within the system convey an implied directionality for the user. In some embodiments, the base comprises a mechanism for attaching the handle pole that distributes forces along the base to maintain stability of the system. In some embodiments, the wheelbase is positioned to maintain the center of mass under the IV pole.

In some aspects, the present invention relates to the geometry of the base to provide flexibility for a variety of motion states, including: navigating bedside, toileting and hallway walking. The geometry enables the system to rotate easily and safely across a variety of tight turning radii typical of the hospital setting. A narrower base geometry relative to existing IV Pole systems increases maneuverability in navigating around narrow spaces in healthcare environments, including doorways and corners. The base is designed with the flexibility to accommodate specialized patient populations, including pediatric and bariatric patients.

The base is designed to provide clinical benefits to the subject, including increased mobility, while also reducing the incidence for foot injury. The geometry of the base creates a more favorable walking experience for the subject, which enhances clinical outcomes, for example decreasing the incidence of deep venous thrombosis (DVT).

In some embodiments, the base comprises features to reduce or improve the bedside footprint of the system. In some embodiments, the base comprises a cast aluminum material. In some embodiments, the base comprises a peripheral metal rim and/or peripheral bumper.

The base is designed to carry heavy vertical loads and resist toppling over. The base is also designed with repairability in mind. In some embodiments, the wheels of the base are replaceable. Aspects of the present invention relate to a design for enabling replacement of the wheels without disassembly or dismantling of the mobile IV stand. In some embodiments, the wheels can be replaced while allowing the mobile IV stand to remain upright. In some embodiments, the base comprises grooves that allow the wheels to provide transient stability as they are being replaced.

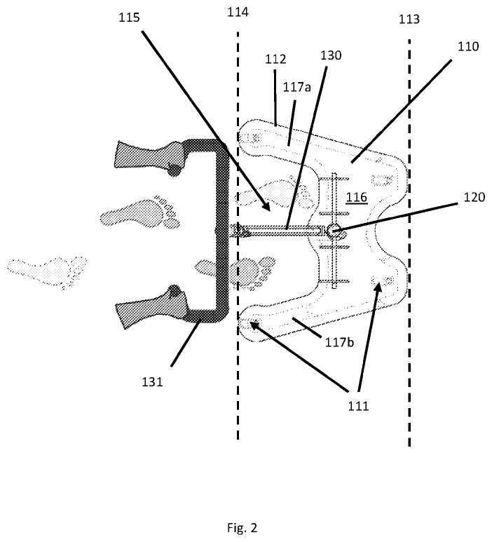

Referring now to FIG. 2, base 110 has a perimeter 112 that may be any desired shape, including generally square, rectangular, trapezoid, circular, semicircular, oval, and the like. Base 110 includes a central region 116 defining a front end 113 of perimeter 112. Base 110 further includes legs 117a and 117b defining a back end 114 of perimeter 112 as well as a cut out, recess or walking space 115 between back end 114 and central region 116 of base 110. In some embodiments, the depth of walking space 115 between back end 114 and central region 116 along perimeter 112 may be between 1 and 24 inches. In some embodiments, the depth of walking space 115 may be at least 2 inches, at least 4 inches, at least 6 inches, at least 8 inches, at least 10 inches, at least 12 inches, at least 14 inches, at least 16 inches, at least 18 inches, at least 20 inches, at least 22 inches, or at least 24 inches.

In some embodiments, base 110 includes at least one wheel 111 on the bottom surface of base 110 underneath each of legs 117a and 117b. In some embodiments, base 110 includes at least one wheel 111 on the bottom surface of base 110 underneath central region 116. In some embodiments, base 110 includes one wheel 110 underneath each of legs 117a and 117b at the back end 114 of base 110, and two wheels 111 underneath central region 116 at the front end 113 of base 110. Base 110 may be composed of materials such as plastics, metal, wood and the like, without limitation. Base 110 may be rigid, or in some embodiments may have one or more regions that are flexible.

IV pole 120 may be a generally vertical pole extending upward from a top surface of central region 116 of base 110. IV pole 120 may be adjustable in height, such as via a telescoping mechanism. IV pole 120 may include a clamp 121 for securing the adjusted length of IV pole 120. In some embodiments, the telescoping mechanism may be triggered and/or locked by a cam lock, spring pins, a latch, and the like. IV pole 120 may be straight along its length, or it may include one or more curves or bends along its length. A top end of IV pole 120 may include one or more attachment structures or hangers 122 for supporting or attaching healthcare equipment such as IV pumps, IV medication bags, foley catheter bags, oxygen tanks, power banks, portable wound dressing suction units, and the like. IV pole 120 may include any number of hangers or attachment structures. IV pole 120 may be attached directly to base 110 or it may be attached to a pole connector 140 which is itself attached to the top surface of base 110.

IV pole 120 may be fixedly attached or removably attached to base 110 or pole connector 140. The mechanism of attachment may be any standard form of attachment, such as a friction fit, threaded engagement, locking spring pins, or the like.

As contemplated herein, handle pole 130 may be separate and independent from IV pole 120, such that they are not in direct contact. In some embodiments, handle pole 130 is connected directly or indirectly to IV pole 120. In some embodiments, handle pole 130 has a bottom end connected directly to base 110, or in other embodiments, handle pole 130 is connected to pole connector 140, which is anchored to the top surface of base 110. Handle pole 130 has a top end or top region which includes a handle 131. Handle pole 130 may be adjustable in length, such as via a telescoping mechanism. Handle pole 130 may include a clamp 132 for securing the adjusted length of handle pole 130. In some embodiments, the telescoping mechanism may be triggered and/or locked by a cam lock, spring pins, a latch, and the like. Handle pole 130 may be straight along its length, or it may include one or more curves or bends along its length.

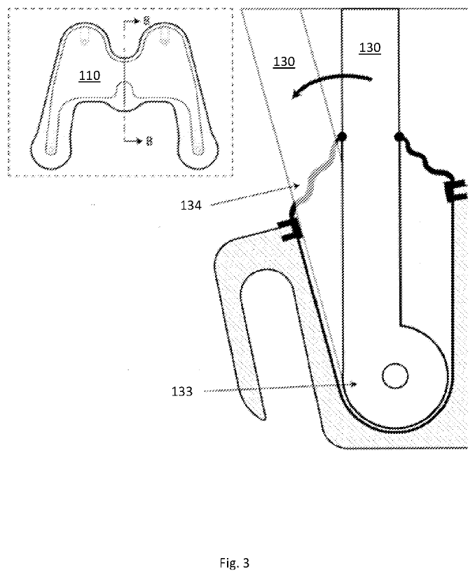

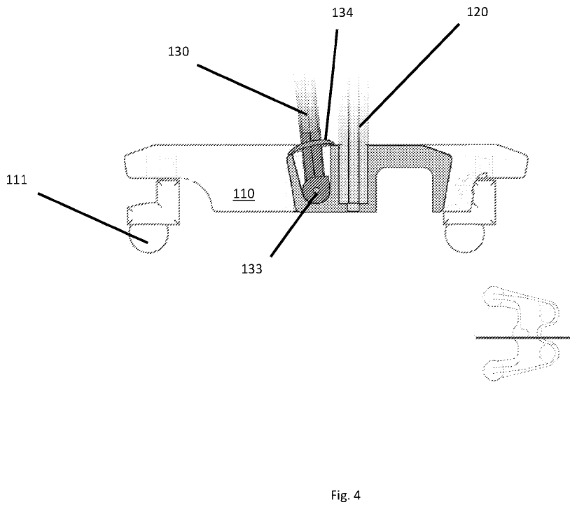

As shown in FIG. 3 and FIG. 4, handle pole 130 may include a hinging mechanism or hinging joint 133 at or near the connection point of handle pole 130 to base 110 or pole connector 140. This hinging joint 133 permits handle pole 130 to pivot or move. In some embodiments, handle pole 130 may be adjustably positioned vertically for easier and more efficient stowage, or at variable angles away from IV pole 120 for a subject to walk with mobile IV stand 100 more comfortably. In some embodiments, the angle of handle pole 130 relative to IV pole 120 may be adjustable to multiple positions at any angle between 0°-90°. In some embodiments, the angle of handle pole 130 relative to IV pole 120 may be adjustable to any angle between 0°-60°. In some embodiments, the angle of handle pole 130 relative to IV pole 120 may be adjustable to any angle between 0°-45°. In some embodiments, the angle of handle pole 130 relative to IV pole 120 may be adjustable to an angle about 0°, or about parallel, about 5°, about 10°, about 15°, about 20°, about 25°, about 30°, about 35°, about 40°, about 45°, about 50°, about 55°, about 60°, about 65°, about 70°, about 75°, about 80°, about 85°, about 90°, or about perpendicular. As contemplated herein, the adjustable angles at which handle pole 130 may be positioned can be releasably secured by any locking mechanism, such as friction clamping, spring pins, ratcheting, and the like. The handle pole 130 locking mechanism may be actuated at the hinging joint 133, from a button, switch or trigger mechanism on handle 131, or from anywhere along the length of handle pole 130. In some embodiments, the button, switch or trigger is covered by a protective cap. In some embodiments, the protective cap protects against infection transmission. In some embodiments, hinging joint 133 may be positioned within a protective boot 134 to prevent unwanted debris from entering and interfering with hinging joint 133.

In some embodiments, handle pole 130 may rotate or pivot left or right of IV pole 120. For example, in some embodiments, hinging joint 133 may also be or include a ball joint for movement with multiple degrees of freedom. Accordingly, in some embodiments handle pole 130 may move, relative to IV pole 120 with at least one degree of freedom or along one axis, at least two degrees of freedom or along two axes, or at least three degrees of freedom or along three axes. In some embodiments, the handle pole may tilt along an axis, providing an operational position wherein the handle pole is tilted away from the IV pole, and a storage position wherein the handle pole is tilted towards the IV pole. In some embodiments, the pole handle tilt may be locked in various positions. In some embodiments, the pole handle comprises a switch or button, wherein activation of the switch or button allows for control of the tilting of the pole handle.

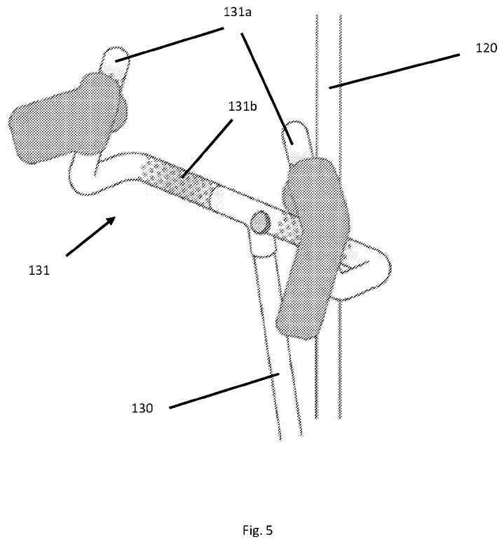

Now referring to FIG. 5, shown is an enlarged view of handle 131. In some embodiments, the at least one handle comprises vertical 131a and/or horizontal 131b grips for the subject to grasp. In some embodiments, handle 131 comprises a material and/or feature to increase the comfort and grip of the handle. For example, handle 131 may comprise knurling, debossed features, embossed features, slits, and/or material coverings such as foam or rubber to increase the ergonomics and grip of handle 131. In some embodiments, vertical grips 131a and/or horizontal grips 131b further comprise a hand-brake for actuably locking wheels 111 between locked and unlocked positions.

Mobile IV stand 100 may be constructed from any type of metal, plastic, polymer, or wood, and combinations of such materials. Components may be manufactured via extrusion, molding, printing or any other standard manufacturing technique. For example, poles 120 and 130 may be extruded metal, such as aluminum. Handle 131 may also be extruded metal and bent as desired. Base 110 may be cast metal or molded plastics, or any other suitable rigid material. In some aspects, the present invention relates to a modification to traditional circular extrusion geometry typically found in IV poles. In some embodiments, the mobile IV stand comprises unique polygonal cross-sections for the extrusion/poles to provide greater stability to the system but still allowing reverse compatibility for all components (cables, oxygen lines, IV lines, and power lines). In some embodiments, the IV pole and/or handle pole comprise a circular geometry. In some embodiments, the IV pole and/or handle pole comprise a non-circular geometry. In some embodiments, the IV pole and/or handle pole comprise an octagonal geometry. In some embodiments, the IV pole and/or handle pole geometry integrates with a clip to assist with tube and cable management. In some embodiments, the clip enables attachment for a foley catheter in a way that does not obstruct gait of the subject, while remaining at the height necessary to drain the bladder of the subject effectively.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

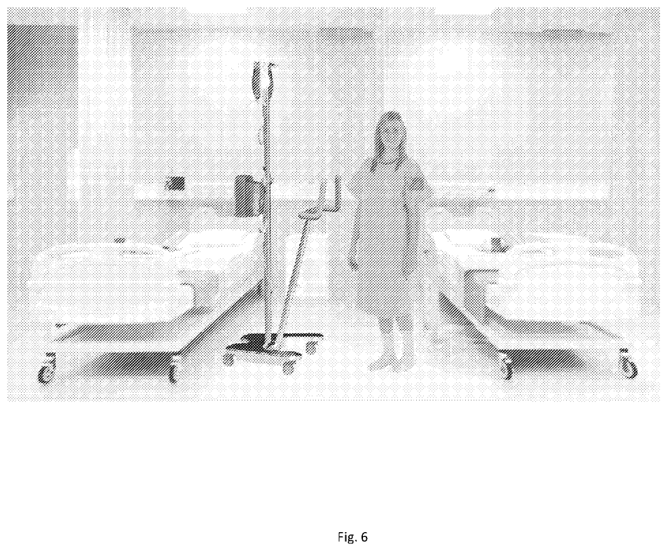

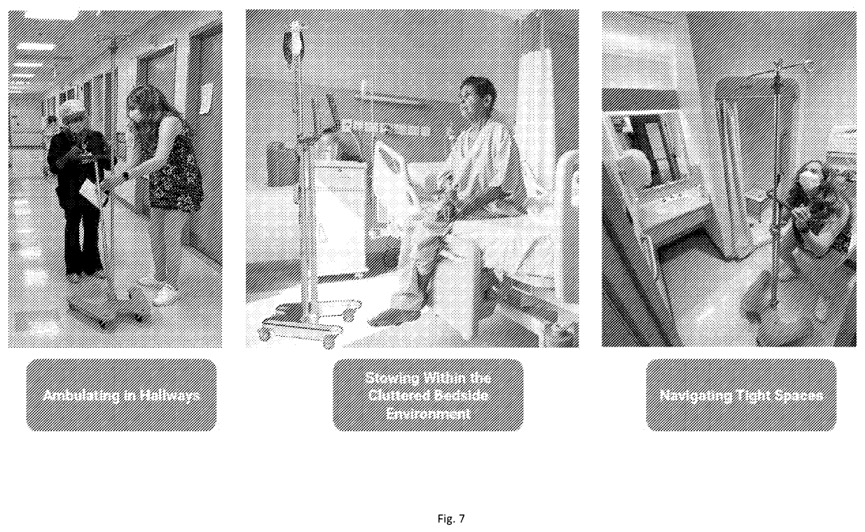

Nearly 100 hours of nurse and team member observation was completed in the hospital to assess patients walking and identify specific pain points. FIG. 6 depicts the mobile IV stand in a hospital setting, with a subject next to the stand for scale. FIG. 7 depicts the mobile IV stand addressing different use states including, ambulating in hallways, stowing away, and navigating tight spaces.

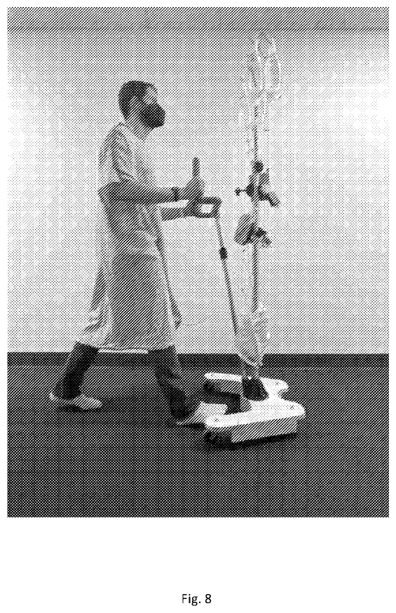

In working towards optimizing the system, the design was reviewed with a clinical nursing team for an inpatient medical/surgical unit at a hospital. There, nine (9) team members were asked to “walk” with the prototype Motivity system on their unit and to evaluate their experience with the system relative to the existing IV pole. FIG. 8 depicts a subject performing normal ambulation with the mobile IV stand.

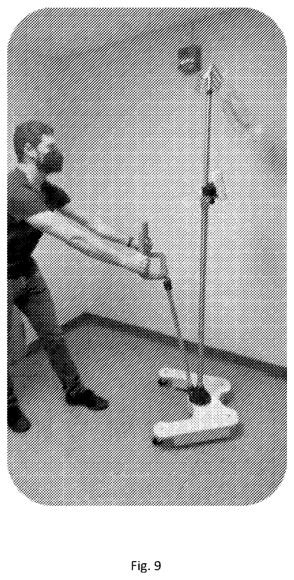

The subjects found the mobile IV stand prototype to be easier to move around, safer, and representative of a more positive walking experience. FIG. 9 displays the safety of the mobile IV stand, as the subject pulls on the handle in an attempt to destabilize the base.

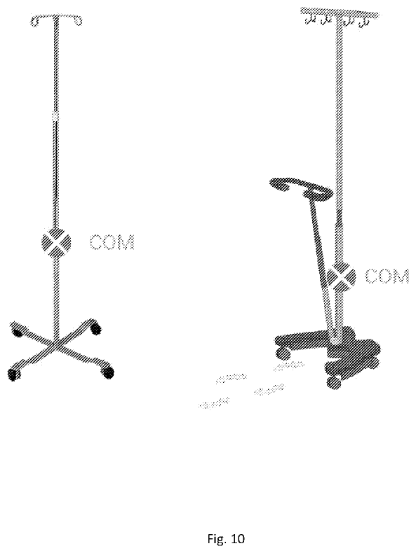

These metrics were compared with prior research from “Sprout Pole”, an IV pole designed for pediatrics. (Parbhu et al., “Differing perspectives: Evaluation of a new IV pole by children and adults.” Journal of Child Health Care 23.4(2019 ): 551-563). FIG. 10 shows the comparison of an existing IV pole vs the mobile IV stand. Included in this depiction is the Center of Mass (COM) for each IV Pole, the mobile IV stand having a lower COM, indicating the mobile IV stand is safer to use.

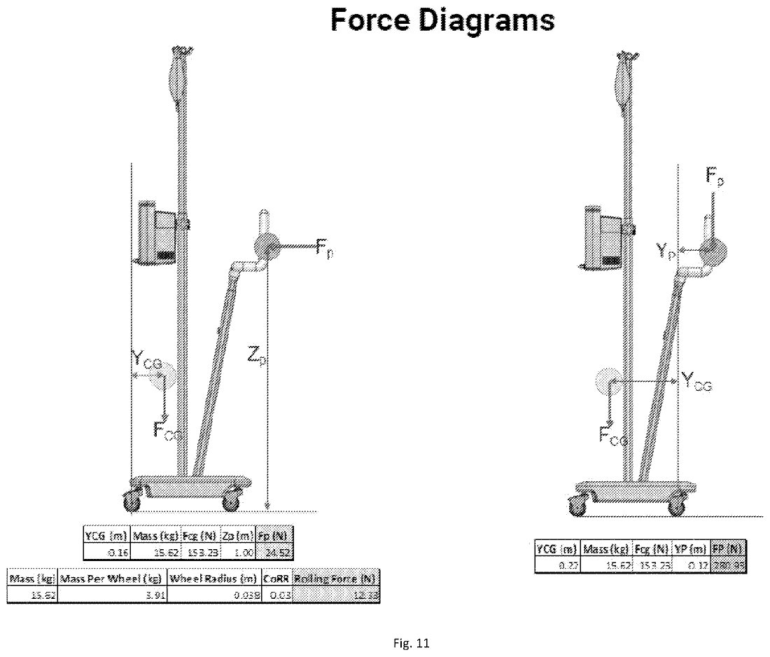

FIG. 11 depicts side-views of the mobile IV stand with force diagrams applied to the system. The figure depicts the Pushing Force (Fp) when applied to the handle in horizontal and vertical directions.

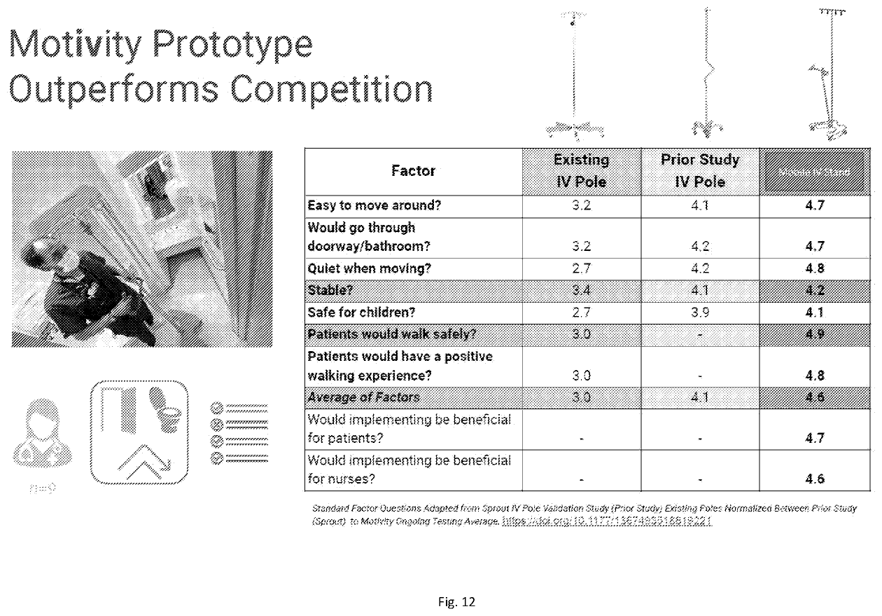

Now referring to FIG. 12, shown is the competitive analysis for the mobile IV stand when compared to existing and prior study IV poles. Overall, the mobile IV stand prototype scored 4.6/5.0, relative to 3.0/5.0 for the existing IV poles.

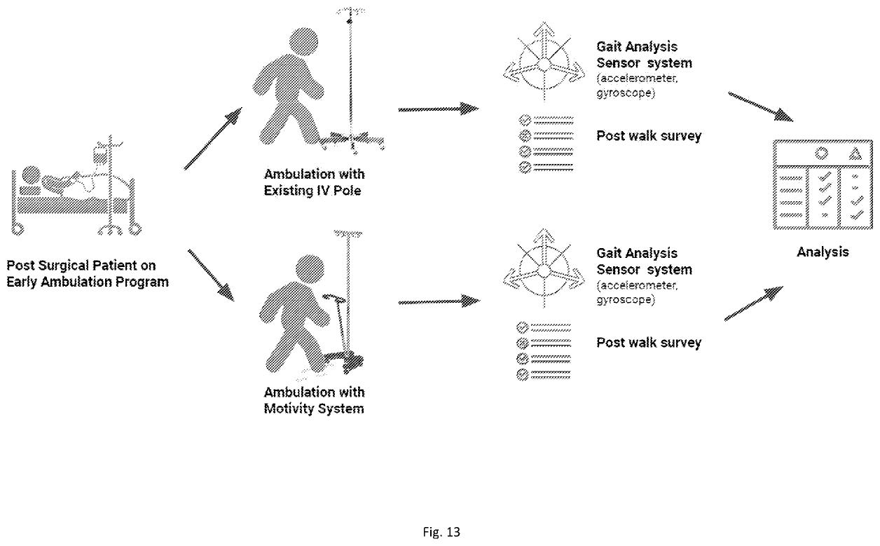

As outlined in FIG. 13, additional studies of the mobile IV stand include subjects ambulating with an existing IV pole and the mobile IV stand as disclosed. Gait analysis using a sensor system (accelerometer, gyroscope) is used to compare the walking gait of the subject between the two systems, investigating the characteristics of the walking gait, foot strikes, and the like.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A mobile IV stand comprising:
a base having a top surface and a bottom surface, the base further including a central region, a front end, and a back end defined by two legs extending from the central region;
at least one wheel on the bottom surface of the base underneath each leg, and at least one wheel on the bottom surface of the base underneath the central region;
an IV pole extending from the top surface of the base; and
a handle pole extending from the top surface of the base;
wherein the handle pole is separate and independent from the IV pole; and
wherein the handle pole moves along at least one axis between at least a first and second lockable position.

2. The mobile IV stand of claim 1, wherein the handle is connected to the base at a hinging joint.

3. The mobile IV stand of claim 2, wherein the hinging joint may be releasably secured by one or more locking mechanisms to arrest movement of the handle pole.

4. The mobile IV stand of claim 3, wherein the one or more locking mechanisms comprises at least one of a friction clamp, spring pin, ratchet, brake and piston.

5. The mobile IV stand of claim 3, further comprising one or more actuators positioned on the handle of the handle pole configured to releasably actuate the locking mechanism.

6. The mobile IV stand of claim 5, wherein the one or more actuators comprise at least one of a button, switch and a trigger.

7. The mobile IV stand of claim 1, wherein the handle pole is configured to pivot along the at least one axis between 0 degrees and 90 degrees relative to the IV pole.

8. The mobile IV stand of claim 1, further comprising one or more hand-grips attached to the handle pole.

9. The mobile IV stand of claim 8, wherein the one or more hand-grips comprise a pair of vertical hand-grips and a pair of horizontal hand-grips.

10. The mobile IV stand claim 8, wherein the one or more hand-grips comprise knurling, debossed features, embossed features, slits, or material coverings such as foam or rubber.

11. The mobile IV stand of claim 1, wherein at least one of the IV pole and handle pole comprise one or more telescoping mechanisms.

12. The mobile IV stand of claim 11, wherein the one or more telescoping mechanisms are locked by one or more locking mechanisms.

13. The mobile IV stand of claim 14, wherein the locking mechanisms comprise at least one of a clamp, cam lock, spring pin, latch, and piston.

14. The mobile IV stand of any preceding claim, wherein each leg has a length between 1 inch and 24 inches.

15. The mobile IV stand of any preceding claim, further comprising a brake configured to releasably lock at least one wheel.

16. The mobile IV stand of any preceding claim, wherein the IV pole comprises one or more attachment elements for healthcare equipment.

17. The mobile IV stand of claim 18, wherein the healthcare equipment is selected from the group consisting of: IV pumps, IV medication bags, foley catheter bags, oxygen tanks, power banks, and portable wound dressing suction units.

18. The mobile IV stand of claim 1, wherein at least one of the IV pole and the handle pole has a recess configured to at least partially house the opposing pole.

19. The mobile IV stand of claim 1, wherein the two legs at least partially define a walking space.

20. A mobile IV stand comprising:
a base comprising a pair of opposing legs and a walking space therebetween;
a plurality of wheels extending from a bottom surface of the base;
an IV pole extending from a top surface of the base along a first axis; and
a handle pole extending from the top surface of the base along a second axis, the handle pole connected to the base at a pivot point and configured to at least partially pivot into an area above the walking space.
